(19) United States
(12) Patent Application Publication
(10) Pub. No.: US 2023/0172666 A1
(43) Pub. Date: Jun. 8, 2023

(54) PRE-OPERATIVE SURGICAL PLANNING

(71) Applicant: Cochlear Limited, Macquarie University, NSW (AU)

(72) Inventors: Antonin RAMBAULT, Mechelen (BE); Guy Fierens, Mortsel (BE)

(21) Appl. No.: 17/917,634

(22) PCT Filed: Mar. 30, 2021
(86) PCT No.: PCT/IB2021/052623

§ 371 Date: Oct. 7, 2022

Related U.S. Application Data
(60) Provisional application No. 63/009,093, filed on Apr. 13, 2020.

(51) Int. Cl. A61B 34/10; H04R 25/00

(52) U.S. Cl. CPC A61B 34/10; H04R 25/606; A61B 2034/104; A61B 2034/105; A61B 2034/107; H04R 2460/13

(57) ABSTRACT

Presented herein are pre-operative surgical planning techniques that enable a user to optimize placement of an implantable component of an implantable medical device in/within the body of a recipient. In particular, a computing device/system is configured to obtain anatomical data associated with the part body of the recipient in which the implantable component is to be implanted. The computing system is configured to analyze the recipient anatomical data to determine one or more suggested implantable placements for the implantable component. The computing device may be configured to predict, at least based on the recipient anatomical data, an estimated outcome for the recipient with the implantable component implanted at a suggested implantable placement.

### BACKGROUND

### Field of the Invention

The present invention relates generally to the pre-operative surgical planning for implantation of implantable components of implantable medical devices.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a pre-operative surgical planning method is provided. The method comprises: obtaining anatomical data associated with a head of a recipient of an implantable auditory prosthesis, wherein the implantable auditory prosthesis includes at least one transducer; determining, based on the anatomical data, one or more suggested implantable placements for the at least one transducer; and displaying, at a display screen, one or more visible indications of the one or more suggested implantable placements.

In another aspect, a method is provided. The method comprises: obtaining anatomical data associated with a recipient of an implantable medical device, wherein the implantable medical device includes at least one implantable component; determining at least one candidate implantable placement for the at least one implantable component; and predicting, at least based on the anatomical data, an estimated outcome for the recipient with the at least one implantable component implanted at the at least one candidate implanted location.

In another aspect, one or more non-transitory computer readable storage media are provided. The one or more non-transitory computer readable storage media comprise instructions that, when executed by a processor, cause the processor to: obtain anatomical data associated with a head of a recipient of an implantable auditory prosthesis, wherein the implantable auditory prosthesis includes at least one implantable actuator; determine, based on the anatomical data, one or more suggested implantable placements for the at least one implantable actuator, wherein each of the one or more implantable placements includes both an implantable location and an implantable orientation for the at least one implantable actuator; and display, at a display screen, one or more visible indications of the one or more suggested implantable placements for the at least one implantable actuator

In another aspect, one or more non-transitory computer readable storage media are provided. The one or more non-transitory computer readable storage media comprise instructions that, when executed by a processor, cause the processor to: obtain acquiring anatomical data representing a bone structure of a head of a recipient of an implantable auditory prosthesis, wherein the implantable auditory prosthesis comprises at least one actuator; determine, based on the anatomical data, at least one suggested implantable location for at least one implantable component of the implantable auditory prosthesis that optimizes sound transmission from the at least one actuator to a cochlea of the recipient; and display, at a display screen, one or more visible indications of the at least one suggested implantable location for the at least one implantable component.

In another aspect, one or more non-transitory computer readable storage media are provided. The one or more non-transitory computer readable storage media comprise instructions that, when executed by a processor, cause the processor to: obtain anatomical data associated with a head of a recipient of an implantable auditory prosthesis, wherein the implantable auditory prosthesis includes at least one implantable sensor; determine, based on the anatomical data, one or more suggested implantable placements for the at least one implantable sensor; and display, at a display screen, one or more visible indications of the one or more suggested implantable placements for the at least one implantable actuator.

### BRIEF DESCRIPTION OF THE DRAWINGS

Embodiments of the present invention are described herein in conjunction with the accompanying drawings, in which:

FIG. 1A is a top view of a totally implantable middle ear auditory prosthesis, with which certain embodiments presented herein may be implemented;

FIG. 1B is a schematic diagram illustrating the totally implantable middle ear auditory prosthesis of FIG. 1A;

FIG. 1C is a functional block diagram of the totally implantable middle ear auditory prosthesis of FIG. 1A;

FIG. 1D is a perspective view of an actuator of the totally implantable middle ear auditory prosthesis of FIG. 1A and a fixation system

FIG. 1E is another perspective view of the actuator and fixation system of FIG. 1D;

FIG. 2 is a schematic diagram illustrating a percutaneous bone conduction device, with which certain embodiments presented herein may be implemented;

FIG. 3 is a schematic diagram illustrating an example medical reconstruction (virtual model) of the head of a recipient generated from recipient anatomical data, in accordance with certain embodiments presented herein;

FIG. 4 illustrates an example visible indication of several suggested placements of an implantable actuator, in accordance with certain embodiments presented herein;

FIG. 5 illustrates an example visible indication of several suggested placements of an implantable actuator, in accordance with certain embodiments presented herein;

FIG. 6 illustrates an example visible indication of several suggested placements of an implantable sensor, in accordance with certain embodiments presented herein;

FIG. 7 illustrates an example visible indication of several suggested placements of an implantable percutaneous abutment, in accordance with certain embodiments presented herein;

FIG. 8 is a schematic diagram illustrating a transcutaneous bone conduction device, with which certain embodiments presented herein may be implemented;

FIG. 9 is a schematic diagram illustrating a retinal prosthesis, with which certain embodiments presented herein may be implemented;

FIG. 10 is a block diagram illustrating a computing device/system configured to implement certain embodiments presented herein;

FIG. 11 is a flowchart of a method, in accordance with certain embodiments presented herein; and

FIG. 12 is a flowchart of another method, in accordance with certain embodiments presented herein.

### DETAILED DESCRIPTION

Presented herein are pre-operative surgical planning techniques that enable a user (e.g., surgeon) to optimize placement (e.g., in terms of location, orientation, etc.) of an implantable component of an implantable medical device in/within the body (e.g., head) of a recipient. In particular, a computing device is configured to obtain anatomical data associated with the part body of the recipient in which the implantable component is to be implanted. This anatomical data is sometimes referred to herein as “recipient anatomical data.” The computing device is configured to analyze the recipient anatomical data to determine one or more suggested implantable placements for the implantable component. The computing device is configured to display one or more visible indications of the one or more suggested implantable placements to the user. The computing device may be configured to predict, at least based on the recipient anatomical data, an estimated outcome for the recipient with the implantable component implanted within a recipient at a suggested implantable placement.

Merely for ease of description, the techniques presented herein are primarily described herein with reference to a totally implantable middle ear auditory prostheses (middle ear implant). However, it is to be appreciated that the techniques presented herein may also be used with a variety of other implantable medical devices. For example, the techniques presented herein may be used with other auditory prostheses, including cochlear implants, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

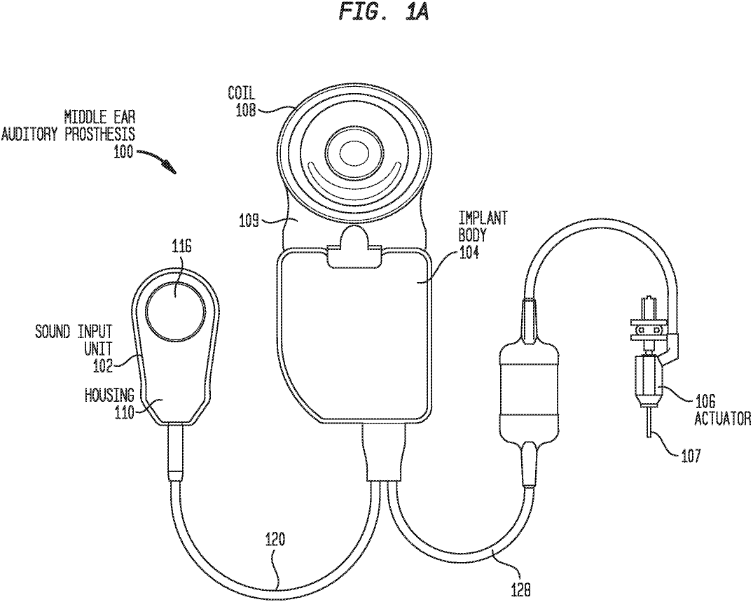

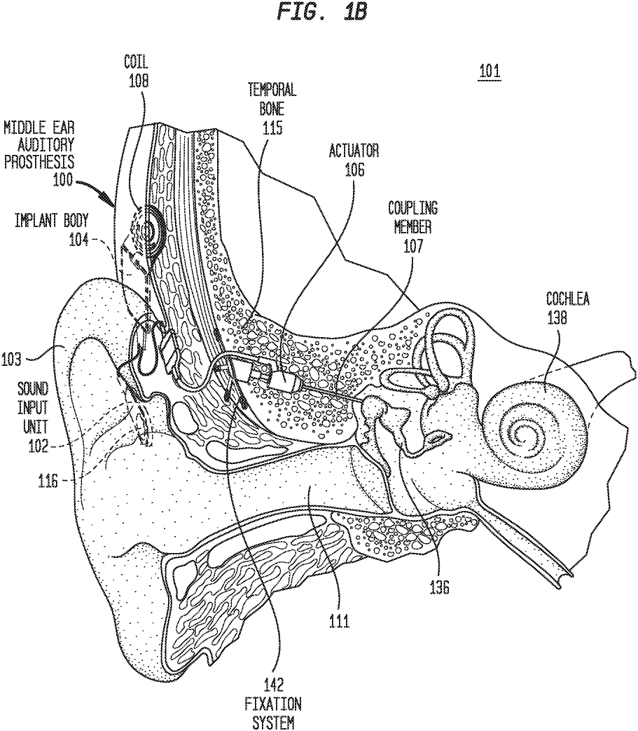

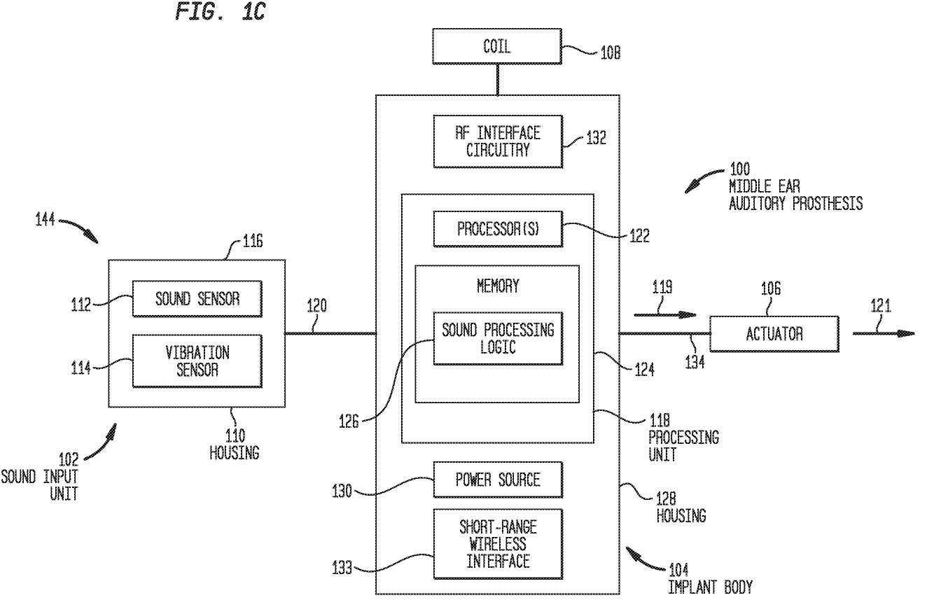

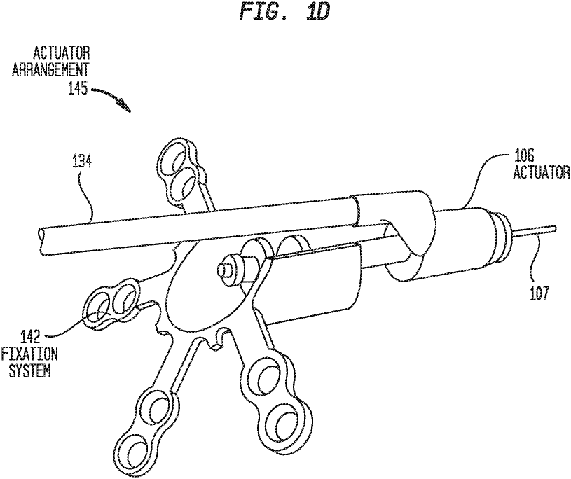

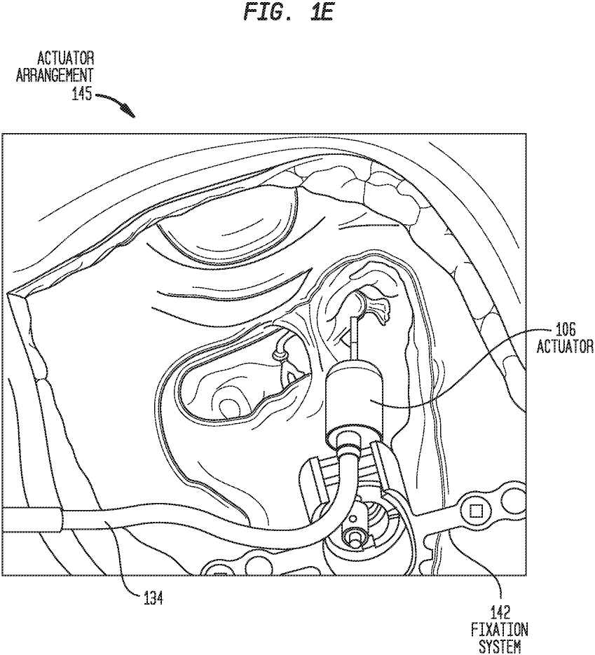

FIG. 1A is a top view of a totally implantable middle ear auditory prosthesis 100, in accordance with certain embodiments presented herein. FIG. 1B is schematic diagram illustrating the middle ear auditory prosthesis 100 of FIG. 1A implanted in a recipient 101, while FIG. 1C is a schematic block diagram of the middle ear auditory prosthesis 100. For ease of description, FIGS. 1A-1C will be described together.

The middle ear auditory prosthesis 100 of FIGS. 1A-1C comprises a sound input module/unit 102, an implant body 104, an actuator 106, and a coil 108, all implanted under the skin/tissue of the recipient 101. The sound input unit 102 comprises a substantially rigid housing 110, in which at least two implantable sensors 112 and 114 are disposed/positioned. The implantable sensor 112 is configured/designed to pick-up (capture) external acoustic sounds, while implantable sensor 114 is configured/designed to pick-up (capture) vibration caused, for example, by body noises. That is, the implantable sensor 112 is a “sound” sensor/transducer that is primarily configured to detect/receive external acoustic sounds, such as an implantable microphone, while the implantable sensor 114 is a “vibration” sensor that is primarily configured to detect/receive internal body noises and vibrations (e.g., vibrations caused by the action of an implantable actuator). The sound sensor 112 and the vibration sensor 114 are sometimes collectively referred to herein as “implantable sensors” 144. As used herein, the actuator 106, sound sensor 112, and vibration sensor 114 are sometimes collectively referred to as “transducers” (i.e., the actuator 106, sound sensor 112, and vibration sensor 114, are each a device that converts variations in a physical quantity (energy) into an electrical signal, or vice versa).

The housing 110 is hermetically sealed and includes a diaphragm 116 that is proximate to the microphone 112. The diaphragm 116 may be unitary with the housing 110and/or may be a separate element that is attached (e.g., welded) to the housing 110. The sound input unit 102 is configured to be implanted within the recipient 101. In one example shown in FIG. 1B, the sound input unit 102 is configured to be implanted within the skin/tissue adjacent to the outer ear 103 of the recipient. In this position, the diaphragm 116 is below the skin of the recipient that is close to the recipient’s ear canal 105. In operation, sound signals that impinge on the skin adjacent to (i.e., on top of) the diaphragm 116 cause the skin adjacent the diaphragm 116, and thus the diaphragm 116 itself, to be displaced (vibrate) in response to the sound signals. The displacement of the diaphragm 116 is detected by the sound sensor 112. In this way, the sound sensor 112, although implanted within the recipient, is able to detect external acoustic sound signals (external acoustic sounds).

In the example of FIGS. 1A-1C, the sound sensor 112 and the vibration sensor 114 may each be electrically connected to the implant body 104 (e.g., in a separate casing connected to the main implant body 104). In operation, the sound sensor 112 and the vibration sensor 114 detect input (sound/vibration) signals (e.g., external acoustic sounds and/or body noises) and convert the detected input signals into electrical signals that are provided to the processing unit 118 (e.g., via lead 120). The processing unit 118 is configured to generate stimulation control signals 119 (FIG. 1C) based at least on the external acoustic sounds and/or the vibrations detected by the sound sensor 112 and/or the vibration sensor 114, respectively.

In the example of FIG. 1B, the processing unit 118 comprises at least one processor 122 and memory 124. The memory 124 includes sound processing logic 126 that, when executed by the at least one processor 122, cause the at least one processor 122 to perform sound processing operations described herein (e.g., convert external acoustic sounds and/or the body noises detected by the sound sensor 112 and/or the vibration sensor 114 into stimulation control signals 119). Memory 124 may comprise any suitable volatile or non-volatile computer readable storage media including, for example, random access memory (RAM), cache memory, persistent storage (e.g., semiconductor storage device, read-only memory (ROM), erasable programmable read-only memory (EPROM), flash memory, etc.), or any other computer readable storage media that is capable of storing program instructions or digital information. The processing unit 118 may be implemented, for example, on one or more printed circuit boards (PCBs).

It is to be appreciated that the arrangement for processing unit 118 in FIG. 1C is merely illustrative and that the techniques presented herein may be implemented with a number of different processing arrangements. For example, the sound processing unit 118 may be implemented with processing units formed by any of, or a combination of, one or more processors (e.g., one or more Digital Signal Processors (DSPs), one or more uC cores, etc.), firmware, software, etc. arranged to perform, for example, the operations described herein.

As shown, the implant body 114 includes a hermetically sealed housing 128 in which the processing unit 118 is disposed. Also disposed in the housing 128 is a power source (e.g., rechargeable battery) 130 and a radio-frequency (RF) interface circuitry 132. Electrically connected to the RF interface circuitry 132 is the implantable coil 108, which is disposed outside of the housing 128. Implantable coil 108 is typically a wire antenna coil comprised of multiple turns of electrically insulated single-strand or multi-strand platinum or gold wire. The electrical insulation of implantable coil 108 is provided by a flexible molding (e.g., silicone molding) 109 (FIG. 1A). In general, the implantable coil 108 and the RF interface circuitry 132 enable the receipt of power and data from an external device (not shown in FIGS. 1A-1C) and, potentially, the transfer of data to an external device. However, it is to be appreciated that various types of energy transfer, such as infrared (IR), electromagnetic, capacitive and inductive transfer, may be used to transfer power and/or data from an external device and, as such, FIG. 1B illustrates only one example arrangement.

As noted, the RF interface circuitry 132 and the implantable coil 108 enable the middle ear auditory prosthesis 100 to receive data/power from and/or transfer data to, an external device. That is, modulated signals transmitted bi-directionally through the inductive link (RF coil 108 and an external) are used to support battery charging, device programming, status queries and user remote control. In certain examples, the external device may comprise an off-the-ear (OTE) unit. In other examples, the external device may comprise a behind-the-ear ear (BTE) unit or a micro-BTE unit, configured to be worn adjacent to the recipient’s outer ear. Alternative external devices could comprise a device worn in the recipient’s ear canal, a body-worn processor, a fitting system, a computing device, a consumer electronic device (e.g., mobile phone communication), etc.

FIG. 1C has been described with reference to use of the RF interface circuitry 132 and the implantable coil 108 for communication with an external device. However, in in certain embodiments, the implant body 104 may also include a short-range wireless interface 133 for communication with external devices. The short-range wireless interface 133 may be, for example, a Bluetooth® interface, Bluetooth® Low Energy (BLE) interface, or other interface making use of any number of standard or proprietary protocols. Bluetooth® is a registered trademark owned by the Bluetooth® SIG.

As noted above, the processing unit 118 generates stimulation control signals 119. The stimulation control signals 119 are provided to the actuator 106 (e.g., via lead 134) for use in delivering mechanical stimulation signals to the recipient. In FIG. 1C, the mechanical stimulation signals (vibration signals or vibration) delivered to the recipient are represented by arrow 121.

In the example of FIG. 1B, the actuator 106 delivers the vibration 121 to the recipient via the ossicular chain (ossicles) 136 (i.e., the bones of the middle ear, which comprise the malleus, the incus and the stapes). That is, the actuator 106 is physically coupled to the ossicles 136 via a coupling member 107 that moves (vibrations) in response to vibration of the actuator 106. The ossicles 136 are positioned in the middle ear cavity 113 and are mechanically coupled between the tympanic membrane 113 and the oval window (not shown) of cochlea 138. In natural hearing, the ossicles 136 serve to filter and amplify sound waves received via the recipient’s ear canal 111.

As shown in FIG. 1B, the actuator 106 is configured to be implanted in the recipient so as to impart motion to (e.g., vibrate) the ossicles 136 or the cochlea fluid directly via, for example, the oval window, the round window, a cochleostomy, etc. In FIG. 1B, the actuator 106 attached to the bone 115 of the recipient via a fixation system 142 (also shown in more detail in FIGS. 1D and 1E). In addition, the actuator 106 is mechanically coupled to the ossicles 136 (e.g., the incus) via a coupling member 107, which may be part of the actuator 106 and/or a separate element attached to the actuator. The actuator 106 and the fixation system 142 are sometimes collectively referred to herein as an “actuator arrangement” 145.

In operation, the actuator 106 is configured to generate vibration 121 based on the stimulation control signals 119 received from the processing unit 118. Since, as noted, the ossicles 136 are coupled to the oval window (not shown) of cochlea 138, vibration imparted to the ossicles 136 by the actuator 106 will, in turn, cause oval window to articulate (vibrate) in response thereto. Similar to the case with normal hearing, this vibration of the oval window sets up waves of fluid motion of the perilymph within cochlea 138 which, in turn, activates the hair cells inside of the cochlea 138. Activation of the hair cells causes appropriate nerve impulses to be generated and transferred through the spiral ganglion cells (not shown) and auditory nerve (not shown) to the brain (also not shown), where they are perceived as sounds.

It is to be appreciated that the arrangement shown in FIG. 1B in which the actuator 106 is mechanically coupled to the ossicles 136 is merely illustrative and that the techniques presented herein may be used with different mechanical stimulation arrangements. For example, in alternative embodiments, the actuator 106 could be coupled directly to the oval window, another opening in the cochlea 138 (e.g., a cochleostomy or the round window), an opening in the recipient’s semicircular canals, the recipient’s skull bone, etc.

The middle ear auditory prosthesis 100 of FIGS. 1A-1E is sometimes referred to as a “totally implantable middle ear auditory prosthesis” because all components of the prosthesis are configured to be implanted under skin/tissue of a recipient. Because all components of the middle ear auditory prosthesis 100 are implantable, the middle ear auditory prosthesis operates, for at least a finite period of time, without the need of an external device. However, as noted, an external device can be used to, for example, are to support battery charging, device programming, status queries, user remote control, etc. of the middle ear auditory prosthesis 100.

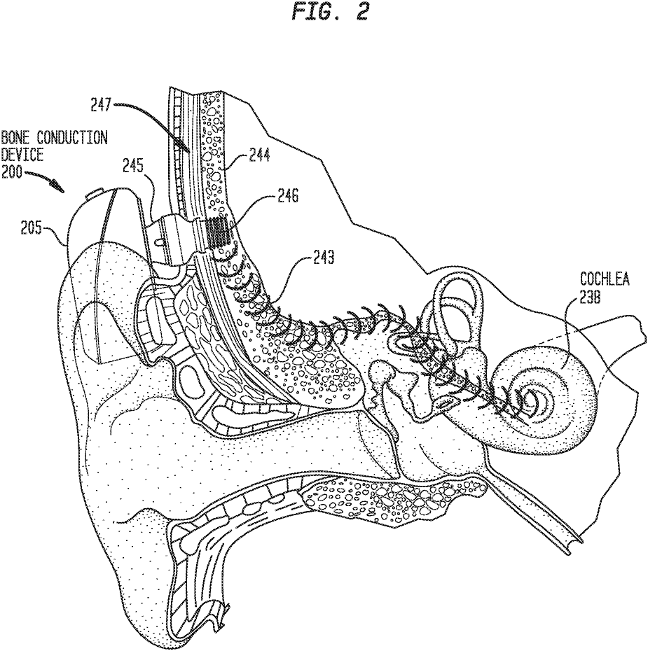

FIG. 2 is a perspective view of a percutaneous bone conduction device 200 that can be used with certain embodiments presented herein. In the example of FIG. 2, the percutaneous bone conduction device 200 is shown positioned behind outer ear 203 of the recipient. The bone conduction device 200 comprises a housing 205 and one or more sound input elements (not shown in FIG. 2) that are configured to receive sound signals. The sound input element(s) can comprise, for example, sound sensors (microphones), telecoils, ports, a wireless interface, etc. The housing 205 of bone conduction device 200 also comprises a sound processor (not shown), a vibrating actuator, and/or the operational components, as needed (e.g., battery, wireless communication circuitry, etc.).

In operation, at least one of the one or more sound input elements receives sound signals. If not already in electrical form, the at least one sound input element converts the received sound signals into electrical signals. The sound processor then converts the electrical signals into actuator control signals that cause the actuator to vibrate. That is, the actuator converts the electrical actuator control signals into mechanical force that imparts vibrations 243 to the skull bone 244 of the recipient. When imparted to the skull bone 244, the vibrations causes motion of the fluid within cochlea 238 of the recipient, which in turn induces a hearing sensation (i.e., enables the recipient to receive the sound signals received at the at least one sound input element 106).

As shown, the bone conduction device 200 further includes a coupling apparatus 245 that attaches the bone conduction device 200 to the recipient. In the example of FIG. 2, the coupling apparatus 245 is attached to an anchor system, namely a percutaneous abutment 246, implanted in the recipient. The percutaneous abutment 246 fixed to the recipient’s skull bone 244 and extends from skull bone 244 through the tissue 247 (e.g., muscle, fat, and/or skin) of the recipient so that coupling apparatus 245 may be attached thereto. Such a percutaneous abutment provides an attachment location for coupling apparatus 245 that facilitates efficient transmission of mechanical force to the skull bone 244.

As noted, implantable medical devices, such as middle ear auditory prosthesis 100 or percutaneous bone conduction device 200, include components that are surgically implanted in a recipient (e.g., surgically implanted beneath the skin, tissue, bone, etc. of a recipient). Currently, it is difficult to determine an “optimal” or “preferred” location for different components. That is, conventional arrangements generally lack the ability to provide a user with a pre-operative indication of whether a particular placement (e.g., in terms of location and/or orientation) of a component is possible for a given recipient and/or whether a particular placement is likely to provide the recipient with favorable outcomes after the surgery. Instead, such determinations are typically made intra-operatively by the surgeon, requiring the surgeon to quickly and accurate assess the recipient’s anatomy based on limited information (e.g., limited visibility).

As noted, is currently difficult to predict the “outcome” for a recipient of an implantable medical device, where the “outcome” refers to how the recipient’s anatomy, physiological process, and/or the implantable component of an implantable medical device will function after a given implantable component is implanted with a particular placement. That is, conventional arrangements generally lack the ability to provide an accurate estimate of how the recipient’s anatomy, physiological process, and/or component of an implantable medical device will function after a component thereof is implanted with a particular placement. These difficulties in predicting the outcome for the recipient increase as the number of implantable components/parts increase, particularly when such implantable components (e.g., implantable sound sensor, implantable vibration sensor, implantable actuator, etc.) are sensitive to anatomical variations, placement, orientation, etc.

Accordingly, presented herein are techniques that are able to provide a user (e.g., surgeon) with preoperative information relating to a proposed placement of one or more implantable components of an implantable medical device, potentially with suggestions for an optimal or preferred placement. In certain embodiments, the techniques that are able to provide the user with an indication of an expected recipient outcome associated with a proposed placement.

More specifically, a system (e.g., computing device, such as a laptop computer, tablet computer, mobile phone, clinical fitting system, surgical system, etc. executing software) in accordance with certain embodiments presented herein is configured to obtain anatomical data associated with the body (e.g., head) of a recipient of an implantable auditory prosthesis or other implantable medical device. As used herein, a “recipient” of an implantable auditory prosthesis or other implantable medical device is a person who has been implanted with an implantable auditory prosthesis or other implantable medical device (e.g., a person who has had a component of the implantable auditory prosthesis or other implantable medical device implanted in his/her body), or a person who is a candidate to be implanted with an implantable auditory prosthesis or other implantable medical device (e.g., a person who may have a component of the implantable auditory prosthesis or other implantable medical device implanted in his/her body sometime in the future).

The anatomical data associated with a body of a recipient of an implantable auditory prosthesis or other implantable medical device, sometimes referred to herein as “recipient anatomical data,” can take a number of different forms and can be obtained in a number of different manners. In certain embodiments, the anatomical data associated with a body of a recipient includes medical imaging obtained via any of a number of different techniques, including X-rays, ultrasounds, computed tomography (CT) scans, magnetic resonance imaging (MRI), echography, nuclear medicine imaging, including positron-emission tomography (PET), etc. In certain examples, the recipient anatomical data includes/indicates (e.g., represents), or can be used to generate a bone density “map” of the head of the recipient, indicating bone thickness and/or density of the bones (e.g., skull bone) as a function of voxel in the head of the recipient. In certain examples, the recipient anatomical data indicates the recipient’s skin thickness and muscle location(s). In certain examples, the recipient anatomical data indicates the location of predetermined surgical landmarks, such as the pinna, nerves (e.g., facial nerve), ossicular chain, etc.

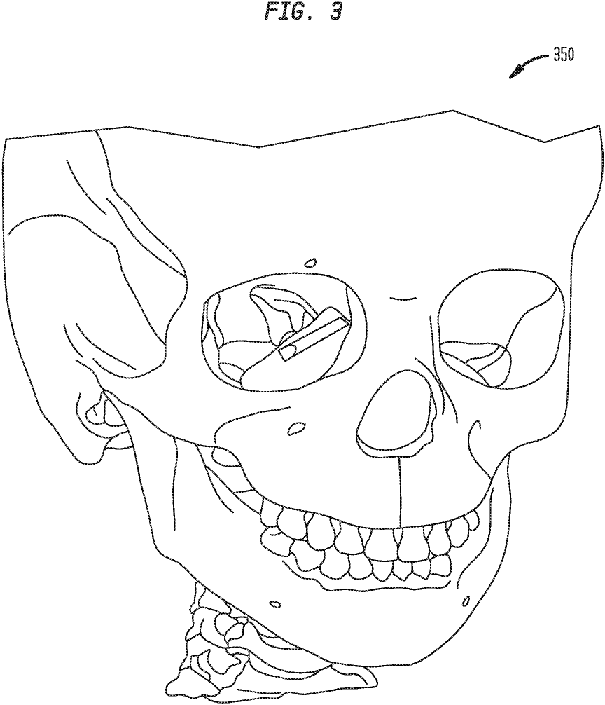

After the recipient anatomical data has been obtained, the recipient anatomical data is analyzed to extract anatomical features of interest (e.g., bone thickness, density, location, etc., ossicular chain location, nerve location, etc.). In certain examples, recipient anatomical data can be used to generate a virtual model (e.g., a two-dimensional or three-dimensional model), sometimes referred to herein as a medical reconstruction, of the recipient’s anatomy for display to a user. FIG. 3 illustrates an example three-dimensional medical reconstruction 350 of a portion of a recipient’s head that can be generated based on recipient anatomical data, in accordance with certain embodiments presented herein. The three-dimensional medical reconstruction 350 could be displayed at a display screen of a computing device (e.g., laptop computer, tablet computer, mobile phone, clinical fitting system, surgical system, etc.).

The system is configured to analyze the recipient anatomical data, including any anatomical features of interest, in order to provide a user with one or more “suggested placements” of one or more implantable components of the implantable medical device. As used herein, a suggested placement of an implantable component can be in terms of a proposed/suggested location of the implantable component within the recipient and/or in terms of a proposed/suggested orientation of the implantable component within the recipient. As described further below, in certain examples, the suggested placement of an implantable component may be added to, and shown as part of the medical reconstruction (virtual model) generated from the recipient anatomical data. Also as described further below, a system can allow a user to change/adjust a placement of an implantable component within a displayed medical reconstruction and provide the user with information related to the adjusted placement.

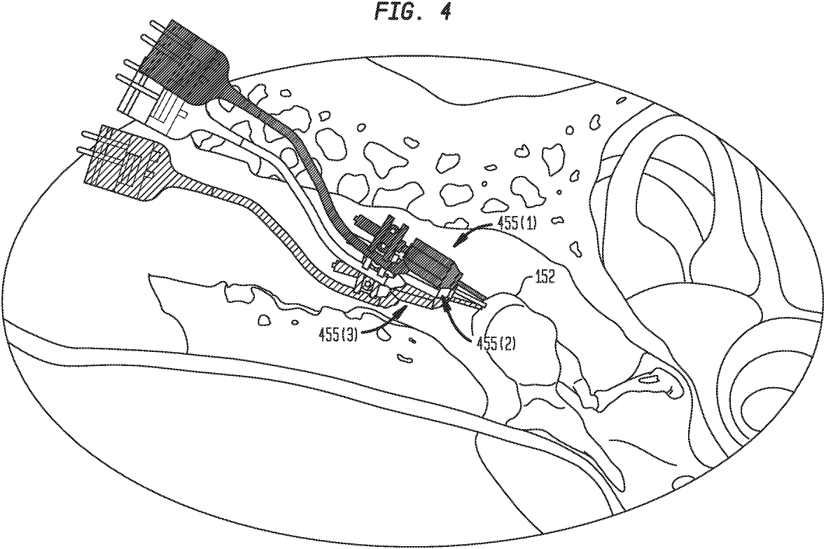

For example, the recipient anatomical can be analyzed to determine one or more suggested placements for a transducer (e.g., actuator or microphone). For purposes of illustration, FIG. 4 will be described with specific reference to the middle ear prosthesis 100 of FIGS. 1A-1E, namely actuator 106. More specifically, FIG. 4 illustrates three (3) suggested middle-ear placements for the actuator 106, with the output shown at a recipient’s incus 152. In FIG. 4, the three suggested placements are referred to as suggested placement 455(1) (i.e., a first suggested placement), suggested placement 455(2) (i.e., a second suggested placement), and suggested placement 455(3) (i.e., a third suggested placement). In the example of FIG. 4, the three suggested placements 455(1)-455(3) propose positioning of the actuator 106 at substantially similar locations within the recipient (e.g., substantially same physical proximity to the incus 152). However, in each of the three suggested placements 455(1)-455(3), the actuator 106 has a different orientation relative to the incus 152. The differences in orientation of the actuator 106 (e.g., the angle at which the actuator will contact the middle ear bone), and thus the differences orientation of the coupling member 107 relative to the incus 152, in each of the suggested placements 455(1)-455(3) changes the mechanics of the physical coupling and how the vibration is transferred to the incus 152 and then to the cochlea 138. Different orientations may be more beneficial for different recipients, depending on their physical characteristics (e.g., as represented in the recipient anatomical data).

FIG. 4 illustrates an example of the information that may be provided to the user, following the analysis of the recipient’s anatomical data, indicating the suggested placements 455(1)-455(3). That is, FIG. 4 illustrates an example medical reconstruction, which includes the suggested placements 455(1)-455(3), which may be provided to a user via a display screen of a computing device. In certain examples, the suggested placements 455(1)-455(3) could be shaded, colored, or otherwise marked to indicate that one placement is preferable to another.

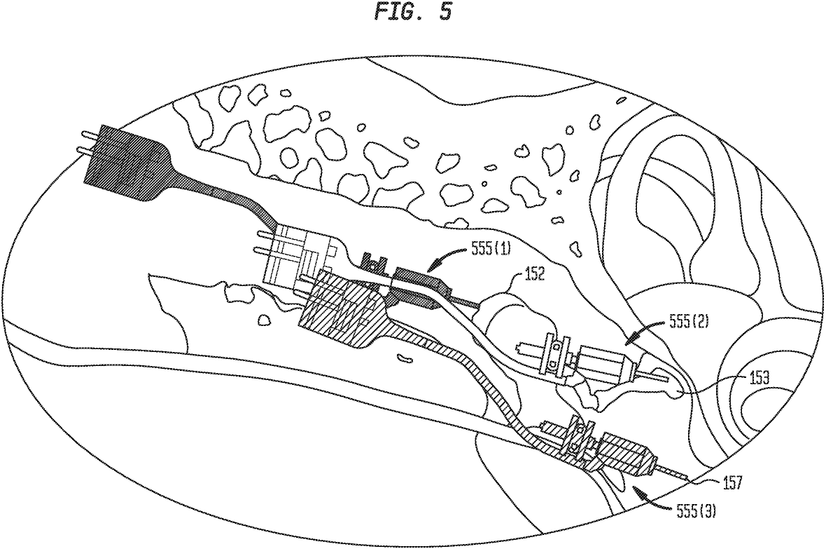

With continued reference to the middle ear prosthesis 100 of FIGS. 1A-1E, FIG. 5 illustrates three (3) further suggested middle-ear placements for the actuator 106. In FIG. 5, the three suggested placements are referred to as suggested placement 555(1) (i.e., a first suggested placement), suggested placement 555(2) (i.e., a second suggested placement), and suggested placement 555(3) (i.e., a third suggested placement). In the example of FIG. 5, the three suggested placements 555(1)-555(3) propose positioning of the actuator 106 at three different locations that are each coupled to the recipient at different locations. More specifically, suggested placement 555(1) proposes that the output of the actuator 106 be coupled to the recipient’s incus 152. However, the second suggested placement 555(2) proposes that the output of the actuator 106 be coupled to the recipient’s stapes 153, while the third suggested placement 555(3) proposes that the output of the actuator 106 be coupled to the recipient’s round window 157.

FIG. 5 illustrates an example of the information that may be provided to the user, following the analysis of the recipient’s anatomical data, indicating the suggested placements 555(1)-555(3). That is, FIG. 5 illustrates an example medical reconstruction that includes the suggested placements 555(1)-555(3), which may be provided to a user via a display screen of a computing device. In certain examples, the suggested placements 555(1)-555(3) could be shaded, colored, or otherwise marked to indicate that one placement is preferable to another.

In general, FIGS. 4 and 5 illustrate that the techniques provide a user with different suggested placements for an implantable actuator. As noted, both the position and the orientation/angle of the actuator can affect the outcomes for the recipient. As such, the techniques presented herein make it possible to provide the user suggested placements that provide a personalized surgical approach for the recipient to ensure that the actuator is implanted in an optimal position and/or orientation based on the recipient’s specific characteristics, including locations of nerves and ossicular chain position. For an implantable actuator, the system may analyze, for example, bone thickness/density, which provides the ability to find the best location for maximizing sound transmission to the targeted cochlea (and minimizing sound transmission to the other) also taking into account bone thickness for fixation and for bone drilling that is needed during the surgery. As used herein, a placement that “optimizes” sound transmission from an implantable actuator to a cochlea refers to a placement that, given the recipient’s specific anatomical characteristics, is likely to provide the relatively most favorable post-surgery vibration transmission pathway between the actuator and the cochlea. Stated differently, the placement that “optimizes” sound transmission is a specific placement, taking into account the recipient’s anatomy, that is expected to provide the most efficient sound path (best vibration transmission characteristics) as a function of voxel (e.g., bone density along the sound conducting pathway), compared/relative to other placements within the recipient).

In FIGS. 4 and 5, the actuator 106 is shown mounted on a fixation system 142. However, it is to be appreciated that the use of a fixation system is merely illustrative and that the techniques presented herein may be implemented with any type of implanted actuator (e.g., floating mass transducers, etc.).

FIGS. 4 and 5 illustrate that the techniques provide a user with different suggested placements for an implantable actuator. It is to be appreciated that similar techniques could be applied to provide a user with different suggested placements for an implantable microphone configured to be coupled to, for example, the middle ear bones of a recipient.

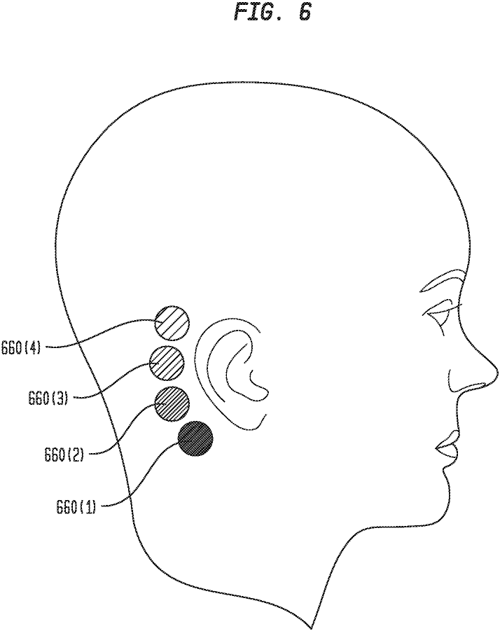

Referring again to the middle ear prosthesis 100 of FIGS. 1A-1E, as noted the prosthesis includes a sound input unit 102 that is implanted in the recipient. FIG. 6 illustrates another embodiment in which the recipient anatomical can be analyzed to determine one or more suggested placements for the sound input unit 102 (e.g., one or more suggested placements for an implantable sound sensor and/or one or more suggested placements for an implantable vibration sensor). As noted, the sound input unit 102 of FIGS. 1A-1C includes both a sound sensor 112 and a vibration sensor 114. However, it is to be appreciated that, in alternative embodiments, a sound input unit could include only a sound sensor or only a vibration sensor. As such, FIG. 6 illustrates an example that can be applied to determine one or more suggested placements for a sound sensor, one or more suggested placements for a vibration sensor, and/or one more suggested placements for a combination of a sound sensor and a vibration sensor.

FIG. 6 illustrates four (4) areas where the sound input unit 102 could be implanted in the recipient, referred to as areas 660(1), 660(2), 660(3), and 660(4). In this example, the analysis of the recipient’s anatomical data reveals that the sound input unit 102 cannot be implanted in area 660(1) because such placement would result in the sound input unit 102 being positioned completely behind the ear of the recipient. Such placement completely behind the ear of the recipient would, in turn, impede the receipt/capture of acoustic sounds.

In addition, the analysis of the recipient’s anatomical data reveals that the sound input unit 102 can be placed in area 660(2), but that such placement is non-optimal (e.g., the ear of the recipient could impede the receipt/capture of acoustic sounds, etc.). However, the analysis of the recipient’s anatomical data reveals that areas 660(3) or 660(4) are the areas most likely to provide an optimal outcome for the recipient. That is, areas 660(3) or 660(4) correspond to areas that optimizes, for the recipient, the capture of external acoustic sound signals (e.g., the ear of the recipient does not impede the receipt/capture of acoustic sounds), areas that position the vibration sensor so as to have a selected sensitivity to body noises, etc. As such, in this example, areas 660(3) or 660(4) would be the suggested placements for the sound input unit 102, based on the recipient’s anatomical data. It is be appreciated that the areas shown in FIG. 6 for implantation of sound input unit 102 are merely illustrative.

As used herein, a placement that “optimizes” the capture of external acoustic sound signals refers to a placement that, given the recipient’s specific anatomical characteristics, is likely to provide the relatively most favorable location to detect external sounds originating outside of the recipient’s body. As used herein, a placement that “optimizes” an expected sensitivity to body noises refers to a placement that, given the recipient’s specific anatomical characteristics, is likely to provide the relatively most favorable location to detect body noises (e.g., sound originating from inside the recipient’s body).

In the embodiments of FIG. 6, following the analysis of the recipient’s anatomical data, a user would be provided with information indicating the suitability of each of the areas 660(1), 660(2), 660(3), and 660(4) for placement of the sound input unit 102. In examples that utilize a virtual model (medical reconstruction), information relating to each area 660(1), 660(2), 660(3), and 660(4) can be presented in the model (e.g., areas 660(1) and 660(2) could be shaded or marked in the same or different manners to indicate that placement of the sound input unit 102 therein is not optimal, areas 660(3) and 660(4) could be shaded or marked as the suggested placement. Areas 660(3) and 660(4) could be shaded or marked differently to indicate that one placement is preferable to the other.

In general, an optimal placement for an implantable sound sensor is one that would enable the sound sensor to best receive external acoustic sounds and, potentially, a placement that reduces the capture of body noises. However, a vibration sensor is designed to capture body noises that are used for sound processing (e.g., noise cancelling) operations. As such, the optimal placement for an implantable vibration sensor is one that would enable the vibration sensor to best receive body noises. In certain embodiments, the optimal placement for a sound input unit with co-located sound and vibrations, such as sound input unit 102, is one that minimizes detection of body noises, although other factors may be considered in other embodiments.

As noted, FIG. 6 has been described with reference to a specific sound input unit that includes both an implantable sound sensor and an implantable vibration sensor. As noted, the techniques presented herein may be used to determine a placement for implantable sound sensors, implantable vibration sensors, and/or units with co-located implantable sound and vibration sensors.

It is also to be appreciated that the techniques presented herein may be used to determine a placement for a variety of different types of implantable sound or vibration sensors. For example, the techniques presented herein may be used to implantable a middle-ear microphone. A middle-ear microphone, sometimes referred to as “TubeMic,” is a type of implantable microphone that is mechanically coupled to a recipient’s ossicular chain. The middle-ear microphone is configured to convert movement of the ossicular chain, which is induced by acoustic signals contacting the ear drum, into electrical signals. Similar to a middle ear actuator, such as actuator 106 above, the optimal placement of a middle-ear microphone would be dependent on both physical location and orientation/angle.

Another type of microphone is designed to sit between bones of the ossicular chain. The techniques presented herein could also be used to determine the optimal placement for such a microphone, where the physical location and orientation/angle may affect microphone sensitivity and frequency behavior.

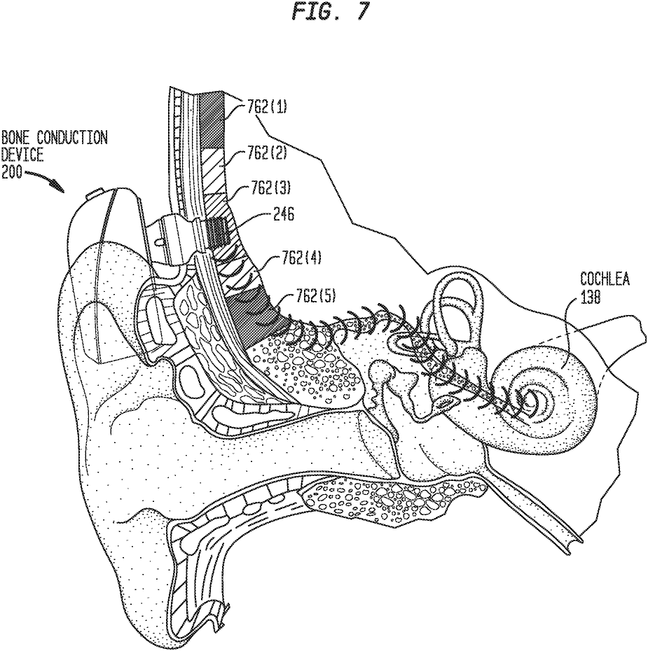

Referring next to the bone conduction device 200 of FIG. 2, as noted the coupling apparatus 245 is attached to an anchor system, namely a percutaneous abutment 246 implanted in the recipient. In certain examples, the recipient anatomical can be analyzed to determine one or more suggested placements for the percutaneous abutment 246, shown in more detail in FIG. 7. More specifically, FIG. 7 illustrates five (5) areas where the percutaneous abutment 246 could be implanted in the recipient, referred to as areas 762(1), 762(2), 762(3), 762(4), and 762(5). In this example, the analysis of the recipient’s anatomical data reveals that the percutaneous abutment 246 cannot be implanted in area 762(1) or in area 762(5). In particular, the percutaneous abutment 246 cannot be implanted in area 762(1) because the recipient anatomical data indicates that this portion of the recipient’s skull bone is too thin for the placement of the abutment 246. The recipient anatomical data indicates that the percutaneous abutment 246 cannot be implanted in area 762(5) because such placement would result in the bone conduction device 200, or at least the microphones of the bone conduction device 200, being positioned behind the ear of the recipient, which would in turn impede the receipt/capture of acoustic sounds.

In addition, the analysis of the recipient’s anatomical data reveals that the percutaneous abutment 246 can be placed in areas 762(2) and 762(4), but that such placement is non-optimal (e.g., the vibration transmission pathway to the cochlea is non-optimal, the skin at such areas is too thick, etc.). However, the analysis of the recipient’s anatomical data reveals that area 762(3) is the area that is most likely to provide an optimal outcome for the recipient (e.g., the skull is sufficiently thick so as to support the abutment, the skin is thin, the microphones are not obstructed by the ear, there is an acceptable, optimal, or the relatively most favorable vibration transmission pathway to the cochlea 138, etc.). As such, in this example, area 762(3) would be the suggested placement for the percutaneous abutment 246, based on the recipient’s anatomical data.

In the embodiments of FIG. 7, following the analysis of the recipient’s anatomical data, a user would be provided with information indicating the suitability of each of the areas 762(1), 762(2), 762(3), 762(4), and 762(5) for placement of the percutaneous abutment 246. In examples that utilize a model, information relating to each area 762(1), 762(2), 762(3), 762(4), and 762(5) can be presented in the model (e.g., areas 762(1) and 762(5) could be shaded or marked to indicate that placement of the abutment 246 therein is not possible, area 762(3) could be shaded or marked as the suggested placement, areas 762(2) and 762(4) could be shaded or marked to indicate that placement of therein is possible, but less than optimal, etc.).

In the context of bone conduction devices, the system in accordance with embodiments presented herein may analyze the recipient’s bone thickness/density, which provides the ability to find the best location for maximizing sound transmission to the targeted cochlea (and minimizing sound transmission to the other) also taking into account bone thickness for fixation (e.g., screws and/or abutment to make sure not to damage the dura) and for bone drilling that is needed during the surgery.

As detailed above, a system in accordance with the techniques presented may analyze the recipient anatomical data and propose any of a number of different suggested placements for an implantable component, such as actuator 106, percutaneous abutment 246, sound input 102, etc. As such, it is to be appreciated that the example suggested placements for actuator 106 shown in FIGS. 4 and 5, for sound input 102 in FIG. 6, and/or for percutaneous abutment 246 in FIG. 7 are merely illustrative.

In summary, FIGS. 4, 5, 6, and 7 illustrate that the techniques presented herein provide implantable component placement recommendations/suggestions to a user (e.g., suggested placement, in terms of location/position, orientation, etc. that maximizes outcome for the specific recipient). The suggested placements are generated from the recipient’s anatomical data and may analyze, for example: (1) the recipient’s bone thickness and bone density, (2) skin thickness, (3) muscle location, (4) nerve location, (4) location of surgical landmarks (e.g., pinna, ossicular chain, etc.), (5), etc. In certain embodiments, the suggested placements can also be based on normative data, including previous clinical data relating to implantation of components in prior recipients (prior recipient data), temporal bone research, bench testing, simulations (e.g., finite element modelling, models based on general anatomy or personalized using recipient’s anatomy), etc. More specifically, the placement optimization could be based on data from prior recipient (e.g., for those recipients, gather a posteriori clinical and anatomical data and try to find a correlation between relevant parameters, such as skin/bone thickness, position relative to outer ear etc.). The optimal placement could also be found using finite element analysis by either reconstructing the anatomy in a model or rework an existing model (e.g., deform the geometry to match the recipient’s anatomy) and based on this anatomy, utilize an iterative approach (e.g., start from a default position and iteratively relocate the implantable component and evaluate how the vibration/sound transmission changes).

The suggested placements could be provided using audible or visible indications, such as a color coding or patterns on/in the medical reconstruction. As noted elsewhere herein, in certain examples, the suggested placements of the implantable components can be placed on a reconstruction of the head or as part of an augmented reality during surgery (with a color code showing best placement or directly illustrate the suggested placement).

As noted, the techniques presented herein provide a user with suggested placements for implantable components of an implantable medical device. In accordance with certain embodiments presented herein, the analysis of suggested placements includes an analysis of an “expected outcome” for the recipient with a suggested placement. That is, a system in accordance with the techniques presented here can provide a user with an estimate of how the recipient’s anatomy, physiological process, and/or the implantable component of an implantable medical device will function after the implantable component is implanted with the placement. The expected outcomes can be determined based on, for example, finite element analysis on a personalized model built from the recipient’s anatomical data or the adaptation/deformation of an existing model to mimic/approach the recipient’s anatomical data, artificial intelligence analysis using previously acquired data and/or finite element analysis predicting outcome, correlation of previously acquired data with the recipient outcome, etc. For example, if a simulation is run based on the recipient’s anatomical data, the expected outcome for a given placement could be computed.

In further embodiments, systems in accordance with techniques presented may obtain post-operative imaging illustrating the final implanted placement of an implantable component. Such systems are configured to correlate the final implanted placement of the implantable component with subjectively or objectively obtain data indicative of recipient outcome(s). The systems presented herein may operate a learning algorithm to generate data, sometimes referred to herein as “prior surgical data,” which can be stored in a central repository. This prior surgical data, if available, could be analyzed as part of the processes to determine suggested placements and/or to determine recipient outcome(s). That is, in certain examples, the techniques presented herein include a predictive model generated based on prior surgical data

As noted above, a system (e.g., computing device) can apply the techniques presented to provide a user with one or more suggested placements of an implantable component of an implantable medical device. In certain embodiments, the one or more suggested placements of an implantable component may be added to, and shown as part of the medical reconstruction (virtual model) generated from the recipient anatomical data, such as shown in FIGS. 4, 5, 6, or 7. In certain such embodiments, a system can apply the techniques to enable a user to change/adjust a placement of an implantable component within the displayed medical reconstruction and provide the user with information related to the adjusted placement.

For example, at least one suggested placement of a first implantable component could be initially displayed to a user within a medical reconstruction. The system could, in certain examples, provide the user with a qualitative or quantitative indication of a recipient outcome with the implantable component at the at least one suggested placement. For example, with implantable actuators, this could be a (window) percentage of efficiency (as the energy transmission to the cochlea), maximum power output. For implantable microphones, an estimate of directionality, sensitivity to ambient sounds and/or body noise could be provided. For bone conduction devices, an estimate of the efficiency (as the energy transmission to the cochlea) and potentially directionality (since the position of the implant conditions the position of the sound processor). Potentially, going forward, all these characteristics could be translated in clinical benefits.

Continuing with the above examples, the user could manipulate the system (e.g., via user inputs) to relocate the first implantable component from the at least one suggested placement to an “adjusted placement” within the medical reconstruction (e.g., move the component based on physiology, anatomy, etc.). After, the first implantable component is placed at the adjusted placement within the medical reconstruction, the system could analyze the adjusted placement, in view of the recipient anatomical data and/or normative data, and provide the user with an indication of the recipient outcome with the implantable component at the adjusted placement. The indication of the recipient outcome with the implantable component at the adjusted placement could be a qualitative or quantitative indication (e.g., advice relating to an impact on the device performance). For example, the indication of the recipient outcome with the implantable component at the adjusted placement could provide a comparison relative to the at least one suggested placement.

In summary of the above, the techniques presented herein obtain recipient anatomical data (e.g., medical imaging) and use the recipient anatomical data to plan the surgery of a recipient of an implantable medical device. For example, pre-operative medical imaging could be loaded in the system so that a user (e.g., surgeon) is able visualize one or more suggested/recommended placements for an implantable component. This visualization could be provided via, for example, highlighted areas within the medical imaging, with a medical reconstruction (virtual model) of the recipient’s anatomy (e.g., 3D model of the implantable component(s) placed on a 3D reconstructed skull of the patient), etc. In examples making use of a model of the recipient’s anatomy, the system can enable the user to virtually adjust/change the placement (e.g., location, orientation, etc.) of the implantable components within the virtual model. The system can provide the user with information/feedback relating to each of the placements of the implantable components within the virtual model. For example, the system can provide the user with indications of whether a given placement is possible, a qualitative indication of the given placement, expected recipient outcomes at a given placement, etc.

In terms of suggested placements, such suggestions could be based on the recipient anatomical data, but also on other data such as, for example, current surgical recommendations. For example, the system e would recognize some landmarks (e.g., auditory canal, nerves, middle ear) and provide the user with recommendations for implant position relative to those landmarks, where the recommendations are based on prior implantation results data. In addition, some research results could be implemented.

For example, for bone conduction devices (bone conduction auditory prostheses), a system in accordance with embodiments presented herein may determine suggested placement(s) by accounting for a particular recipient’s bone density as a function of voxel, and suggest placing an implantable component of the bone conduction device (e.g., transducer, such as an implantable microphone or actuator, percutaneous abutment, anchor system, etc.) at a location that is estimated to optimize sound transmission (including accounting for sound path). For a middle ear auditory prosthesis, a system in accordance with embodiments presented herein may determine suggested placement(s), including which structure (e.g., which ossicular bone, cochlea opening, area of skull bone, etc.) to which an implantable actuator should be mechanically coupled and the orientation of the implantable actuator relative to that structure, by accounting for a recipient’s nerve structure and considering a suitable angle of approach for the surgeon. For an implantable sound sensor, such as an implantable microphone, a system in accordance with embodiments presented herein may determine suggested placement(s) by accounting for a particular recipient’s bone density as a function of voxel, and placing the implant so as to maximize acoustic pickup, while minimizing body noise pickup. As noted, the recipient’s outcome may also be predicted on the same or other data using, for example, finite element analysis, artificial analysis, previously acquired data, etc. It is to be appreciated that these considerations for determining a suggest placement for implantable components are merely illustrative and that other factors could be used to determine the suggested placement(s).

Embodiments of the techniques presented herein have been primarily described above with reference to an example middle ear auditory prosthesis 100 and/or an example percutaneous bone conduction device 200. However, as noted elsewhere herein, it is to be appreciated the embodiments presented herein may be used for surgical planning of the implantation of implantable components of other types of implantable medical devices, such as implantable components of other auditory prostheses, including cochlear implants, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

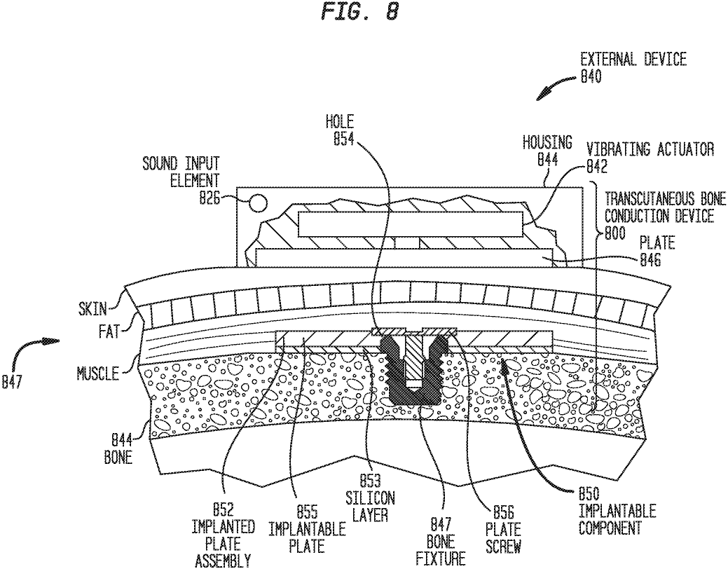

For example, FIG. 8 depicts an exemplary embodiment of a transcutaneous bone conduction device 800 having an implantable component 850 that may be implanted based on the techniques presented herein. The transcutaneous bone conduction device 800 of FIG. 8 is a passive transcutaneous bone conduction device comprising an external device 840 that includes a vibrating actuator 842. Vibrating actuator 842 is located in housing 844 of the external component, and is coupled to plate 846. Plate 846 may be in the form of a permanent magnet and/or in another form that generates and/or is reactive to a magnetic field, or otherwise permits the establishment of magnetic attraction between the external device 840 and the implantable component 850 sufficient to hold the external device 840 against the skin of the recipient.

In an exemplary embodiment, the vibrating actuator 842 is a device that converts electrical signals into vibration. In operation, sound input element 826 converts sound into electrical signals. Specifically, the transcutaneous bone conduction device 800 provides these electrical signals to vibrating actuator 842, or to a sound processor (not shown) that processes the electrical signals, and then provides those processed signals to vibrating actuator 842. The vibrating actuator 842 converts the electrical signals (processed or unprocessed) into vibrations. Because vibrating actuator 842 is mechanically coupled to plate 846, the vibrations are transferred from the vibrating actuator 842 to plate 846. Implanted plate assembly 852 is part of the implantable component 850, and is made of a ferromagnetic material that, in certain embodiments, may be in the form of a permanent magnet, that generates and/or is reactive to a magnetic field, or otherwise permits the establishment of a magnetic attraction between the external device 840 and the implantable component 850 sufficient to hold the external device 840 against the skin of the recipient. Accordingly, vibrations produced by the vibrating actuator 842 of the external device 840 are transferred from plate 846 across the skin to plate 855 of plate assembly 852. This may be accomplished as a result of mechanical conduction of the vibrations through the skin, resulting from the external device 840 being in direct contact with the skin and/or from the magnetic field between the two plates. These vibrations are transferred without penetrating the skin with a solid object such as an abutment as detailed above with respect to a percutaneous bone conduction device.

As may be seen, the implanted plate assembly 852 is substantially rigidly attached to bone fixture 846 in this embodiment. In this regard, implantable plate assembly 852 includes through hole 854 that is contoured to the outer contours of the bone fixture 846. This through hole 854 thus forms a bone fixture interface section that is contoured to the exposed section of the bone fixture 846. In an exemplary embodiment, the sections are sized and dimensioned such that at least a slip fit or an interference fit exists with respect to the sections. Plate screw 856 is used to secure plate assembly 852 to bone fixture 846. As can be seen in FIG. 8, the head of the plate screw 856 is larger than the hole through the implantable plate assembly 852, and thus the plate screw 856 positively retains the implantable plate assembly 852 to the bone fixture 846.

As noted, in the example of FIG. 8, implantable component 850 is implanted in the recipient so that the implanted plate assembly 852 (e.g., implantable plate 855) can transfer vibration to the skull of the recipient, which in turn is relayed to the cochlea. The pre-operative surgical planning techniques presented herein may be used to determine an optimal implantable location for the implantable component 850. For example, a system in accordance with embodiments presented herein may determine suggested placement(s) for implantable component 850 by accounting for a particular recipient’s bone density as a function of voxel, and suggest placing the implantable component 850 at a location that optimizes sound transmission (including accounting for sound path) to the cochlea. In addition, the external device 840 is magnetically coupled to the implantable component 850 and, as such, the location of the external device 840 is set by the placement of the implantable component 850. A system in accordance with embodiments presented herein may determine suggested placement(s) for implantable component 850 by also accounting for an estimated position of the external device 840 to ensure that the sound input element 826 can adequately capture acoustic sounds. It is to be appreciated that these considerations for determining a suggest placement for implantable component 850 are merely illustrative and that other factors could be used to determine the suggested placement(s).

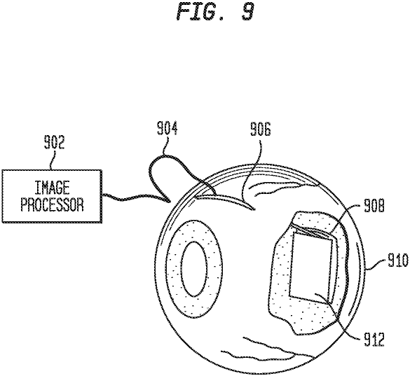

FIG. 9 depicts an exemplary embodiment of a neural prosthesis in general, and a retinal prosthesis and an environment of use thereof, in particular, having components that may be implanted based on the techniques presented herein. In the example of FIG. 9, the retinal prosthesis includes a retinal prosthesis sensor-stimulator 908 configured to be positioned proximate the retina 910. In an exemplary embodiment, photons entering the eye are absorbed by a microelectronic array of the sensor-stimulator 908 that is hybridized to a glass piece 912 containing, for example, an embedded array of microwires. The glass can have a curved surface that conforms to the inner radius of the retina. The sensor-stimulator 908 can include a microelectronic imaging device that can be made of thin silicon containing integrated circuitry that convert the incident photons to an electronic charge.

An image processor 902 is in signal communication with the sensor-stimulator 908 via, for example, a cable 904 which extends through surgical incision 906 through the eye wall (although in other embodiments, the image processor 902 is in wireless communication with the sensor-stimulator 908). The image processor 902 processes the input into the sensor-stimulator 908, and provides control signals back to the sensor-stimulator 908 so the device can provide processed and output to the optic nerve. That said, in an alternate embodiment, the processing is executed by a component proximate to or integrated with the sensor-stimulator 908. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light / image capture device (e.g., located in / on a BTE device or a pair of glasses, etc.), while, as noted above, in some embodiments, the sensor-stimulator 908 captures light / images, which sensor-stimulator is implanted in the recipient.

As noted, in the example of FIG. 9, sensor-stimulator 908 is implanted in the recipient. The pre-operative surgical planning techniques presented herein may be used to determine an optimal implantable location for the implantable component 908. For example, a system in accordance with embodiments presented herein may determine suggested placement(s) for sensor-stimulator 908 by accounting for a particular recipient’s anatomy and suggest placing the sensor-stimulator 908 that optimally captures photons entering the eye. It is to be appreciated that these considerations for determining a suggest placement for sensor-stimulator 908are merely illustrative and that other factors could be used to determine the suggested placement(s).

In the interests of compact disclosure, any disclosure herein of a microphone or sound capture device corresponds to an analogous disclosure of a light / image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor / image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

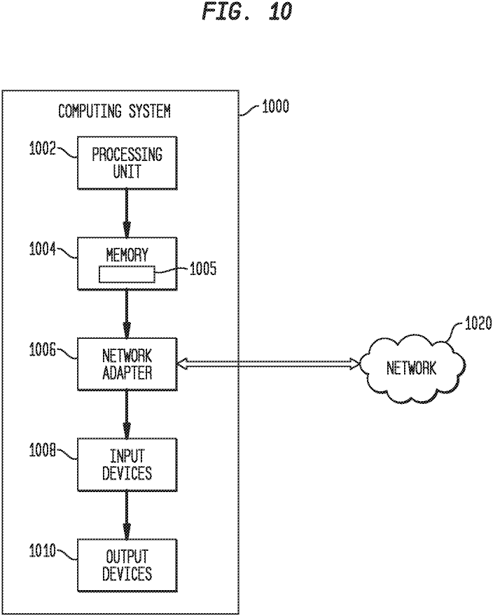

FIG. 10 illustrates an example of a suitable computing system 1000 with which one or more of the disclosed examples can be implemented. Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The computing system 1000 can be a single virtual or physical device operating in a networked environment over communication links to one or more remote devices. The remote device can be an auditory prosthesis (e.g., an auditory prosthesis), a personal computer, a server, a router, a network personal computer, a peer device or other common network node.

In its most basic configuration, computing system 1000 includes at least one processing unit 1002 and memory 1004. The processing unit 1002 includes one or more hardware or software processors (e.g., Central Processing Units) that can obtain and execute instructions. The processing unit 1002 can communicate with and control the performance of other components of the computing system 1000.

The memory 1004 is one or more software or hardware-based computer-readable storage media operable to store information accessible by the processing unit 1002. The memory 1004 can store, among other things, instructions executable by the processing unit 1002 to implement applications or cause performance of operations described herein, as well as other data. The memory 1004 can be volatile memory (e.g., RAM), non-volatile memory (e.g., ROM), or combinations thereof. The memory 1004 can include transitory memory or non-transitory memory. The memory 1004 can also include one or more removable or non-removable storage devices. In examples, the memory 1004 can include RAM, ROM, EEPROM (Electronically-Erasable Programmable Read-Only Memory), flash memory, optical disc storage, magnetic storage, solid state storage, or any other memory media usable to store information for later access. In examples, the memory 1004 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 1004 can include wired media such as a wired network or direct-wired connection, and wireless media such as acoustic, RF, infrared and other wireless media or combinations thereof. In certain embodiments, the memory 1004 comprises pre-operative surgical planning logic 1005 that, when executed, enables the processing unit 1002 to perform aspects of the techniques presented.

In the illustrated example, the system 1000 further includes a network adapter 1006, one or more input devices 1008, and one or more output devices 1010. The system 1000 can include other components, such as a system bus, component interfaces, a graphics system, a power source (e.g., a battery), among other components.

The network adapter 1006 is a component of the computing system 1000 that provides network access (e.g., access to at least one network 1020). The network adapter 1006 can provide wired or wireless network access and can support one or more of a variety of communication technologies and protocols, such as ETHERNET, cellular, BLUETOOTH, near-field communication, and RF (Radiofrequency), among others. The network adapter 1006 can include one or more antennas and associated components configured for wireless communication according to one or more wireless communication technologies and protocols.

The one or more input devices 1008 are devices over which the computing system 1000 receives input from a user. The one or more input devices 1008 can include physically-actuatable user-interface elements (e.g., buttons, switches, or dials), touch screens, keyboards, mice, pens, and voice input devices, among others input devices.

The one or more output devices 1010 are devices by which the computing system 1000 is able to provide output to a user. The output devices 1010 can include, displays, speakers, and printers, among other output devices.

It is to be appreciated that the arrangement for computing system 1000 shown in FIG. 10 is merely illustrative and that aspects of the techniques presented herein may be implemented at a number of different types of systems/devices. For example, the computing system 1000 could be a laptop computer, tablet computer, mobile phone, surgical system, etc.

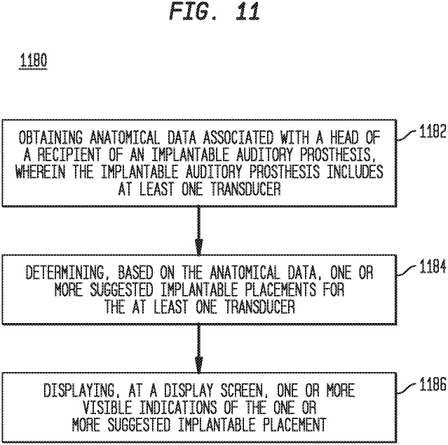

FIG. 11 is a flowchart of a pre-operative surgical planning method 1180 in accordance with embodiments presented herein. Method 1180 begins at 1182 where a computing device/system obtains anatomical data associated with a head of a recipient of an implantable auditory prosthesis, where the implantable auditory prosthesis includes at least one transducer. At 1184, the computing system determines, based on the anatomical data, one or more suggested implantable placements for the at least one transducer. At 1184, the computing system displays, at a display screen, one or more visible indications of the one or more suggested implantable placements.

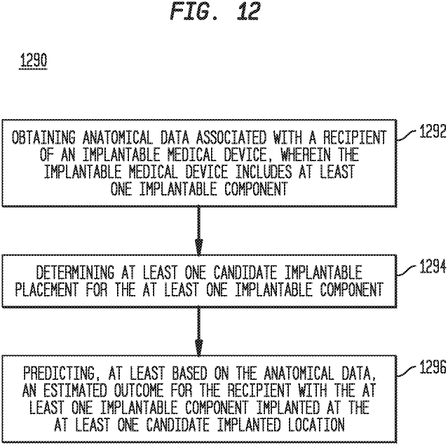

FIG. 12 is a flowchart of a method 1290 in accordance with embodiments presented herein. Method 1290 begins at 1292 where a computing device/system obtains anatomical data associated with a recipient of an implantable medical device, where the implantable medical device includes at least one implantable component. At 1294, the computing system determines at least one candidate implantable placement for the at least one implantable component. At 1296, the computing system predicts, at least based on the anatomical data, an estimated outcome for the recipient with the at least one implantable component implanted at the at least one candidate implanted location.

It is to be appreciated that the embodiments presented herein are not mutually exclusive and that the various embodiments may be combined with another in any of a number of different manners.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A pre-operative surgical planning method, comprising:
obtaining anatomical data associated with a head of a recipient of an implantable auditory prosthesis, wherein the implantable auditory prosthesis includes at least one transducer;
determining, based on the anatomical data, one or more suggested implantable placements for the at least one transducer; and
displaying, at a display screen, one or more visible indications of the one or more suggested implantable placements.

2. The method of claim 1, wherein obtaining anatomical data associated with the head of the recipient comprises:
obtaining medical imaging of the head of the recipient.

3. The method of claim 1, wherein determining, based on the anatomical data, one or more suggested implantable placements for the at least one transducer includes:
determining a bone density map of the head of the recipient from the anatomical data.

4. The method of claim 1, wherein the at least one transducer comprises at least one implantable actuator configured to deliver vibration to the head of the recipient.

5. The method of claim 4, wherein determining one or more suggested placements comprises:
determining one or more implantable locations for the at least one implantable actuator that are expected to optimize, for the recipient, sound transmission from the least one implantable actuator to a cochlea of the recipient.

6. The method of claim 5, wherein determining one or more implantable locations for the at least one implantable actuator that are expected to optimize, for the recipient, sound transmission from the at least one implantable actuator to a cochlea of the recipient, includes:
determining the one or more implantable locations for the at least one implantable actuator while accounting for a bone density of the recipient as a function of voxel.

7. The method of claim 5, further comprises:
determining one or more implantable locations and one or more implantable orientations for the at least one implantable actuator that are expected to optimize, for the recipient, sound transmission from the least one implantable actuator to the cochlea of the recipient.

8. The method of claim 4, wherein determining one or more suggested placements comprises:
determining which one of a plurality of structures of the recipient to which the at least one implantable actuator is to be coupled to for delivery of mechanical stimulation to the recipient in order to optimize, for the recipient, sound transmission from the least one implantable actuator to a cochlea of the recipient.

9. (canceled)

10. The method of claim 1, wherein the at least one transducer is an implantable sound sensor, and wherein determining one or more implantable placements comprises:
determining, based on the anatomical data, at least one implantable location for the implantable sound sensor that optimizes, for the recipient, capture of external acoustic sound signals.

11. The method of claim 1, wherein the at least one transducer is an implantable vibration sensor, and wherein determining one or more implantable placements comprises:
determining, based on the anatomical data, an implantable location for the implantable vibration sensor that optimizes, for the recipient, an expected sensitivity of the vibration sensor to body noises.

12. The method of claim 1, wherein the at least one transducer comprises an implantable sound sensor and an implantable vibration sensor co-located in an implantable sound input unit, and wherein determining one or more implantable placements comprises:
determining, based on the anatomical data, an implantable location for the implantable sound input unit that balances an ability of the vibration sensor to capture of body noises and an ability of the implantable sound sensor to capture of external acoustic sound signals.

13. The method of claim 1, further comprising:
determining, for at least one of the one or more suggested implantable placements, an estimated outcome for the recipient associated with the at least one of the one or more suggested implantable placements; and
providing an indication of the estimated outcome for recipient associated with the at least one of the one or more suggested implantable placements.

14. The method of claim 1, wherein determining, based on the anatomical data, one or more suggested implantable placements for the at least one transducer includes:
generating a virtual model of the head of the recipient based on the anatomical data.

15. The method of claim 14, wherein providing one or more visible indications of the one or more suggested implantable placements to a user comprises:
displaying the one or more suggested implantable placements within the virtual model of the head of the recipient.

16. The method of claim 15, further comprising:
receiving one or more user inputs setting an adjusted implantable placement of the at least one transducer; and
displaying the adjusted implantable placement of the at least one transducer within the virtual model of the head of the recipient.

17. The method of claim 16, further comprising:
providing an indication of an estimated outcome for recipient associated with the adjusted implantable placement.

18-37. (canceled)

38. One or more non-transitory computer readable storage media comprising instructions that, when executed by a processor, cause the processor to:
obtain anatomical data associated with a recipient of an implantable medical device, wherein the implantable medical device includes at least one implantable component;
determine at least one candidate implantable placement for the at least one implantable component; and
predict, at least based on the anatomical data, an estimated outcome for the recipient with the at least one implantable component implanted at the at least one candidate implanted location.

39. The one or more non-transitory computer readable storage media of claim 38, wherein the instructions executable to predict the estimated outcome for the recipient with the at least one implantable component implanted at the at least one candidate implanted location comprise instructions executable to:
predict the estimated outcome based on the anatomical data and based on normative data.

40. The one or more non-transitory computer readable storage media of claim 38, wherein the instructions executable to obtain anatomical data associated with the recipient comprise instructions executable to:
obtain medical imaging of one or more body parts of the recipient.

41. The one or more non-transitory computer readable storage media of claim 38, wherein the instructions executable to determine the at least one candidate implantable placement for the at least one implantable component comprise instructions executable to:
determine, based on the anatomical data, at least one suggested implantable placement for the at least one implantable component.

42. The one or more non-transitory computer readable storage media of claim 38, further comprising instructions executable to:
display, at a display screen, one or more visible indications of the estimated outcome for the recipient.

43. The one or more non-transitory computer readable storage media of claim 38, further comprising instructions executable to:
display, at a display screen, one or more visible indications of the at least one candidate placement for the implantable component.

44. The one or more non-transitory computer readable storage media of claim 43, wherein the instructions executable to display one or more visible indications of the at least one candidate placement for the implantable component comprise instructions executable to:
display the at least one candidate placement within a medical reconstruction of a part of the body of the recipient.
